(19) United States
(12) Patent Application Publication
(10) Pub. No.: US 2025/0387130 A1
(43) Pub. Date: Dec. 25, 2025

(54) RETRIEVAL DEVICES, ASSEMBLIES, SYSTEMS, AND RELATED METHODS

(71) Applicant: Boston Scientific Scimed, Inc., Maple Grove, MN (US)

(72) Inventors: Cristopher OVIEDO MIRANDA, Alajuela (CR); Laura Emily SABBAN, Norton, MA (US); Juan Pablo ORTIZ GARCIA, Heredia (CR); Gian Franco LOO FUCHS, San Jose (CR); Jose Roberto SOTO GUZMAN, Alajuela (CR); Julian FUENTES CASTRO, La Unión (CR); Ana Lucia PEREZ SABORÍO, San Jose (CR); James J. SCUTTI, Norwell, MA (US)

(73) Assignee: Boston Scientific Scimed, Inc., Maple Grove, MN (US)

(21) Appl. No.: 19/240,630

(22) Filed: Jun. 17, 2025

Related U.S. Application Data
(60) Provisional application No. 63/662,185, filed on Jun. 20, 2024.

(51) Int. Cl. A61B 17/221; A61B 17/00

(52) U.S. Cl. CPC A61B 17/221; A61B 2017/00477; A61B 2017/2212

(57) ABSTRACT

Medical devices, systems, and related methods for capturing objects within a subject (e.g., patient) are described. The medical device includes a sheath, a shaft, and a retrieval device coupled to the shaft. At least a portion of the shaft is movably disposed within the sheath. The retrieval device includes a plurality of wires coupled to a distal end of the shaft, and a retention member coupled to distal ends of the plurality of wires. Movement of the shaft relative to the sheath is configured to release at least one wire of the plurality of wires from the retention member.

### CROSS-REFERENCE TO RELATED APPLICATION(S)

This application claims the benefit of priority to U.S. Provisional Application No. 63/662,185, filed on Jun. 20, 2024, which is incorporated by reference herein in its entirety.

### TECHNICAL FIELD

Various aspects of the disclosure relate generally to retrieval devices, assemblies, systems, and related methods. Examples of the disclosure relate to retrieval devices, assemblies, systems, and related methods, for example, concerning a medical instrument with an expandable basket or expandable element, among other aspects.

### BACKGROUND

Advancements in medical devices, assemblies, systems, and related methods, have enabled users to perform increasingly complex medical procedures. One challenge in the field of medical procedures is associated with extracting objects from a patient, such as a biliary stone from a bile duct of the patient. These procedures often require multiple instruments to extract the object, which may cause various procedural issues and/or increase the risk of injury to the patient. In some aspects, the object(s) are captured with an expandable retrieval basket delivered through a sheath or tube, but the object(s) can be too big to be removed from the patient through the sheath or tube. In these aspects, it can be difficult to release the object(s) from the retrieval basket, for example, such that the retrieval basket can be removed from the patient. It would be useful to improve medical extraction devices, assemblies, systems, and related methods to mitigate the risk of procedural issues and/or injury to the patient during medical procedures that involve extracting objects.

### SUMMARY

Examples of this disclosure relate to, among other things, retrieval devices, assemblies, systems, and related methods.

According to one example, a medical device may include a sheath, a shaft, and a retrieval device coupled to a distal end of the shaft. The shaft may include at least a portion movably disposed within the sheath. The retrieval device may include a plurality of wires having proximal ends coupled to the distal end of the shaft, and a retention member coupled to distal ends of the plurality of wires. The retrieval device may be movable between at least a closed state and an open state, may be configured to radially compress into the closed state when positioned within the sheath, and may be configured to radially expand into the open state when positioned outside of the sheath. Movement of the shaft relative to the sheath may be configured to release one or more of the plurality of wires from the retention member of the retrieval device.

Any medical device or medical system described herein may include any of the following features. The retention member may include a cannula that includes a body having a lumen extending therein along a longitudinal axis, in which the plurality of wires are coupled at least partially within the lumen. The medical device may include a plug configured to be received within the lumen of the cannula, in which distal ends of the plurality of the wires are coupled within the lumen of the cannula between the plug and the body of the cannula. The distal ends of the plurality of wires may engage the body of the cannula at an angle relative to the longitudinal axis of the cannula. The distal ends of the plurality of wires may be bent over a distalmost end of the body of the cannula.

The plurality of wires may radially expand into the open state to define a cavity configured to capture an object therein. The plurality of wires may include a first set of wires and a second set of wires, in which distal ends of the first set of wires are coupled together, and in which distal ends of the second set of wires are coupled together. The plurality of wires may include a third set of wires and a fourth set of wires, in which distal ends of the third set of wires are coupled together, and in which distal ends of the fourth set of wires are coupled together.

The retention member may include a coil disposed around the plurality of wires. Proximal translation of the shaft relative to the sheath may cause the coil to release one or more of the plurality of wires. The coil may be disposed around distal ends of the plurality of wires in a z-shaped configuration. The retention member may be welded to at least one of the plurality of wires. The retention member may be welded to at least one of the first set of wires or the second set of wires of the plurality of wires. The medical device may include a tether having a distal portion coupled to the retention member and a proximal portion coupled to one of the sheath and the shaft. The tether may include at least one of a polymer material and a metal material.

According to another example, a medical device may include a sheath, a shaft, and a retrieval device coupled to a distal end of the shaft. The retrieval device may include a plurality of wires, in which proximal ends of the plurality of wires are coupled to the distal end of the shaft, and a distal tip coupled to distal ends of the plurality of wires. The distal tip may include a body having a lumen extending therein along a longitudinal axis between a proximal end and a distal end, and at least one slot extending through the body along the longitudinal axis from one of the proximal end of the body and the distal end of the body.

Any medical device or medical system described herein may include any of the following features. The slot may extend from the distal end of the body proximally toward the proximal end of the body. The slot may include a first slot and a second slot, in which the first slot extends distally from the proximal end of the body, and in which the second slot extends proximally from the distal end of the body toward a distal end of the first slot.

According to another example, a medical device may include a sheath, a shaft, and a retrieval device coupled to a distal end of the shaft. The shaft may include at least a portion movably disposed within the sheath. The retrieval device may include a plurality of wires and a hypotube. The plurality of wires may include proximal ends coupled to the distal end of the shaft, and distal end coupled to the hypotube. The plurality of wires may include a first set of wires and a second set of wires, in which distal ends of the first set of wires are secured within a distal portion of the hypotube, and in which distal ends of the second set of wires are secured within a proximal portion of the hypotube. The retrieval device may be movable between at least a closed state and an open state, may be configured to radially compress into the closed state when positioned within the sheath, and may be configured to radially expand into the open state when positioned outside of the sheath.

Any medical device or medical system described herein may include any of the following features. The first set of wires may be welded to the distal portion of the hypotube, and the second set of wires may be secured to the hypotube via a crimp.

Any of the examples described herein may have any of these features in any combination.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 depicts a medical device, according to aspects of the disclosure.

FIGS. 2A and 2B depict the medical device of FIG. 1, according to aspects of the disclosure.

FIG. 3 depicts another medical device, according to aspects of the disclosure.

FIG. 4 depicts yet another medical device, according to aspects of the disclosure.

FIG. 5 depicts a further other medical device, according to aspects of the disclosure.

FIG. 6 depicts a distal portion of another medical device, according to aspects of the disclosure.

FIG. 7 depicts a distal portion of yet another medical device, according to aspects of the disclosure.

FIG. 8 depicts a distal portion of a further other medical device, according to aspects of the disclosure.

FIGS. 9A and 9B depict a distal portion of another medical device, according to aspects of the disclosure.

FIG. 10 depicts another medical device, according to aspects of the disclosure.

FIG. 11 depicts another medical device, according to aspects of the disclosure.

FIG. 12 depicts another medical device, according to aspects of the disclosure.

### DETAILED DESCRIPTION

Examples of the disclosure include devices, assemblies, systems, and methods for providing a medical instrument including a retrieval device having expandable members that are selectively expandable for collecting and removing a target object from a target treatment site within a patient.

As used herein, the term “distal” refers to a portion farthest away from a user when introducing a device into a patient, and the term “proximal” refers to a portion closest to the user when placing the device into the subject. The terms “comprises,” “comprising,” “includes,” “including,” “has,” “having,” or any other variation thereof, are intended to cover a non-exclusive inclusion, such that a process, method, article, or apparatus that comprises a list of elements does not necessarily include only those elements, but may include other elements not expressly listed or inherent to such process, method, article, or apparatus. The term “exemplary” is used in the sense of “example,” rather than “ideal.” As used herein, the terms “about,” “substantially,” and “approximately,” indicate a range of values within +/−10% of a stated value.

Examples of the disclosure may relate to devices, assemblies, systems, and methods for performing various medical procedures and/or treating portions of the biliary duct, large intestine, small intestine, cecum, esophagus, any other portion of the gastrointestinal tract, and/or any other suitable patient anatomy (collectively referred to herein as a “target treatment site”). Various examples described herein include single-use or disposable medical devices, although some embodiments may include one or more reusable components of the device. Reference will now be made in detail to examples of the disclosure described above and illustrated in the accompanying drawings. Wherever possible, the same reference numbers will be used throughout the drawings to refer to the same or like parts.

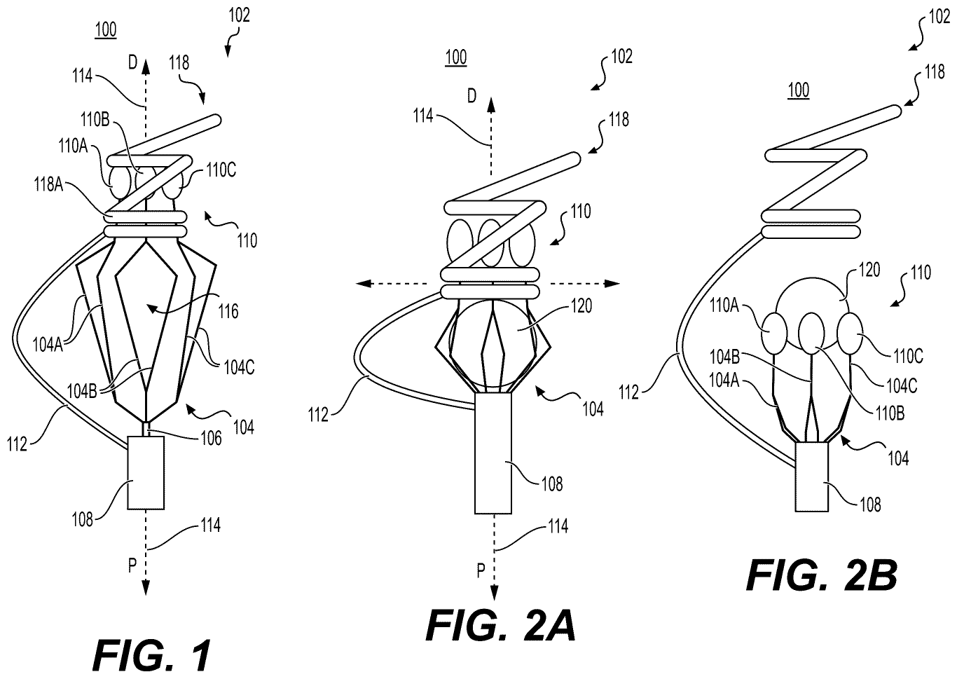

FIG. 1 shows a medical device 100, according to aspects of the disclosure. For example, although not shown, medical device 100 may be delivered through a working channel or other lumen of an endoscope, duodenoscope, gastroscope, colonoscope, ureteroscope, bronchoscope, and/or various other delivery systems to a target treatment site. The medical device 100 may include a handle (not shown) having one or more ports configured to receive and/or control one or more medical instruments therein and/or one or more actuators. As shown, the medical device 100 includes a retrieval device 102 coupled to a distal end of a shaft 106. As shown, the retrieval device 102 may include a plurality of wires 104 (hereinafter, “the wires 104”) extending between a proximal end and a distal end of the retrieval device 102. The wires 104 may include proximal ends coupled to the distal end of the shaft 106 at the proximal end of the retrieval device 102, and distal ends coupled to a retention member 118 at a distal end of the retrieval device 102.

In some aspects, the retrieval device 102 may be movable between at least a closed state and an open state. The retrieval device 102 may radially compress into the closed state and radially expand into the open state. For example, the wires 104 of the retrieval device 102 may radially compress into the closed state when positioned within a sheath 108 of the medical device 100, and the wires 104 may radially expand when positioned outside of the sheath 108. The shaft 106 may be at least partially disposed in the sheath 108. The shaft 106 and the sheath 108 may move (e.g., translate and/or rotate) relative to each other. For example, the distal end of the shaft 106 may translate distally or proximally relative to the sheath 108, which may cause the retrieval device 102 to translate relative to the sheath 108. In the closed state, the retrieval device 102 may translate distally through the sheath 108 and radially expand (e.g., into the open state) when the distal end of the shaft 106 extends distal of the distal end of the sheath 108. In the open state, the retrieval device 102 may define a cavity 116 for capturing one or more objects therein (e.g., a biliary stone in a bile duct).

In some aspects, the wires 104 may include, for example, one or more wire filaments splitting into multiple wires in a substantially bulbous shape and converging at ends thereof. For example, the wires 104 may include three, four, five, six, seven, eight or more wires, which collectively form the substantially bulbous shape. The wires 104 may include one or more flexible and/or shape-memory materials (e.g., Nitinol) that allow the retrieval device 102 to self-expand into the open state (e.g., when positioned outside of or distal to a sheath or lumen). Additionally, or alternatively, one or more portions of the retrieval device 102 may be formed as or otherwise include a net and/or mesh that facilitates retrieval of smaller fragments of material. In other implementations, the retrieval device 102 may be configured to expand manually into the open state.

In some aspects, the wires 104 of the retrieval device 102 may be grouped into two or more sets of wires 104. For example, the wires 104 of the retrieval device 102 may be grouped into a first set 104A of wires 104, a second set of wires 104B, and a third set of wires 104C. Two or more distal portions of the wires 104 may be secured together, for example, to define respective sets of the wires 104 using one or more fixation or coupling processes (e.g., crimping, soldering, welding, adhesive bonding, etc.). In one example, the wires 104 of the retrieval device 102 may include six wires, such that each of the first set 104A, second set 104B, and the third set 104C include two of the wires 104 having respective distal portions coupled together to define respective sets. Although it should be understood that each set of the wires 104 may have more or fewer wires defining respective sets.

As shown, the retrieval device 102 may include a plurality of welds 110 (hereinafter, “the welds 110”) respectively coupling distal ends of the wires 104 that form each of the first set 104A, the second set 104B, and the third set 104C of wires 104. For example, the welds 110 may include a first weld 110A secured to the first set 104A of wires 104, a second weld 110B secured to the second set 104B of wires 104, and a third weld 110C secured to the third set 104C of wires 104. The welds 110 may be formed, for example, by using a weld process that provides a volume of material for joining two or more distal ends of the wires 104. The welds 110 may help to at least partially define a closed distal end of the cavity 116 of the retrieval device 102 (e.g., in the open state).

In some aspects, it may be useful to open the closed distal end of the cavity 116 of the retrieval device 102, for example, to selectively release one or more objects captured therein. As shown, the retrieval device 102 may include a retention member 118 secured to the wires 104 and/or the welds 110. The retention member 118 may include, for example, a coil that is disposed around the distal ends of the wires 104 and/or the welds 110. The retention member 118 may apply a retention force, for example, in a radially inward direction relative to a longitudinal axis 114 of the retrieval device 102 to help constrain movement of the wires 104 and/or the welds 110. The retention member 118 may help to bias the wires 104 and/or the welds 110 in the radially inward direction relative to the longitudinal axis 114, for example, to at least partially defines the closed distal end of the cavity 116 (e.g., in the open state of the retrieval device 102). As shown, the retention member 118 may include one or more proximal loops 118A and one or more distal loops 118B. The proximal loops 118A may be positioned proximal to the distal loops 118B relative to the longitudinal axis 114. In one example, the proximal loops 118A may be disposed around the wires 104 proximal of the welds 110, and the distal loops 118B may be disposed around the welds 110 distal of the proximal loops 118A. The proximal loops 118A and/or the distal loops 118B of the retention member 118 may be fixedly or permanently secured to the wires 104 and/or the welds 110 using one or more fixation processes, such as welding or soldering.

In some aspects, the medical device 100 may include a tether 112. The tether 112 may help to prevent separation of the retention member 118 from the medical device 100 during extraction medical procedures. The tether 112 may include one or more suture materials, polymer fiber materials, and/or metal materials (e.g., Nitinol, stainless steel, metal alloy, cobalt-chromium alloy, etc.). As shown, the tether 112 may include a proximal portion coupled to the shaft 106 and/or the sheath 108, and a distal portion coupled to the retention member 118. In one example, the tether 112 includes a proximal end secured to a distal portion of the sheath 108 and a distal end secured to a proximal portion of the retention member 118. In another example, the tether 112 may be secured to the shaft 106, the sheath 108 and/or the retention member 112 using one or more fixation processes (e.g., tying, crimping, soldering, welding, adhesive bonding, etc.). In another example, the tether 112 may be secured to the shaft 106, the sheath 108 and/or the retention member 112 using a crimp collar or other coupling mechanism. As shown in FIG. 2B and as discussed below, the tether 112 may be secured to the retention member 118 after releasing the object 120. In these aspects, the tether 112 may help to maintain a connection between the retention member 118 and the shaft 106, thus helping to prevent the retention member from being released (e.g., in a body lumen of a patient during medical procedures).

In some aspects, it may be useful to cause the retention member 118 to separate or release at least one of the wires 104 and/or at least one of the welds 110 of the retrieval device 102, for example, to induce failure in the medical device 100 during medical procedures and release object(s) captured therein. FIG. 2A shows one example of the medical device 100 of FIG. 1 capturing an object 120, according to aspects of the disclosure. FIG. 2B shows one example of the medical device 100 of FIG. 1 releasing the object 120 therein, according to aspects of the disclosure. For example, movement of the shaft 106 relative to the sheath 108 (FIG. 1) may cause movement of the retrieval device 102 relative to the sheath 108 (e.g., between the open state and the closed state), which in turn may cause the retrieval device 102 to capture (FIG. 2A) and/or to release (FIG. 2B) the object 120 therein.

In one example, proximal translation of the shaft 106 relative to the sheath 108 causes proximal translation of the retrieval device 102 relative to, and at least partially into, the sheath 108 (e.g., proximal retraction of the retrieval device 102). As shown, the proximal ends of the wires 104 may retract proximally into the sheath 108 in response to proximal translation of the shaft 106 relative to the sheath 108. In turn, this may cause the retrieval device 102 to radially compress relative to the longitudinal axis 114, for example, radially compress the wires 104 towards the closed state and into contact with the object 120 therein. As the retrieval device 102 continues moving proximally, the object 120 may move proximally relative to and into contact with the distal end of the sheath 108. This contact may impede movement of the object 120 proximally beyond the distal end of the sheath 108 relative to the retrieval device 102, and the retrieval device 102 may continue to move proximally relative to the object 120. As shown in FIG. 2A, this may cause the object 120 move into contact with the retention member 118 causing the object 120 to impart a movement force on the retention member 118 in a distal direction relative to the sheath 108. In some aspects, the movement force imparted by the object 120 may be greater than or equal to the retention force of the retention member 118 on the wires 104 and/or the welds 110, which may cause the retention member 118 to release the at least one of the wires 104 and/or at least one of the welds 110. If the movement force is less than the retention force, proximal translation of the retrieval device 102 relative to the sheath 108 may stop, for example, due to contact between the object 120 and distal end of the sheath 108.

In alternative implementations, the retention member 118 may include a plate, for example, bent in a z-shape and including one or more holes formed in the plate. In one example, one or more of the wires 104 may be threaded through the hole(s) of the retention member 118. In one example, proximal translation of the shaft 106 relative to the sheath 108 causes at the wires 104 to move radially inward relative to the retention member 118, for example, to allow the wires 104 to pass through the hole(s) of the retention member 118 (e.g., z-shaped plate) and in turn release one or more objects captured in the retrieval device 102.

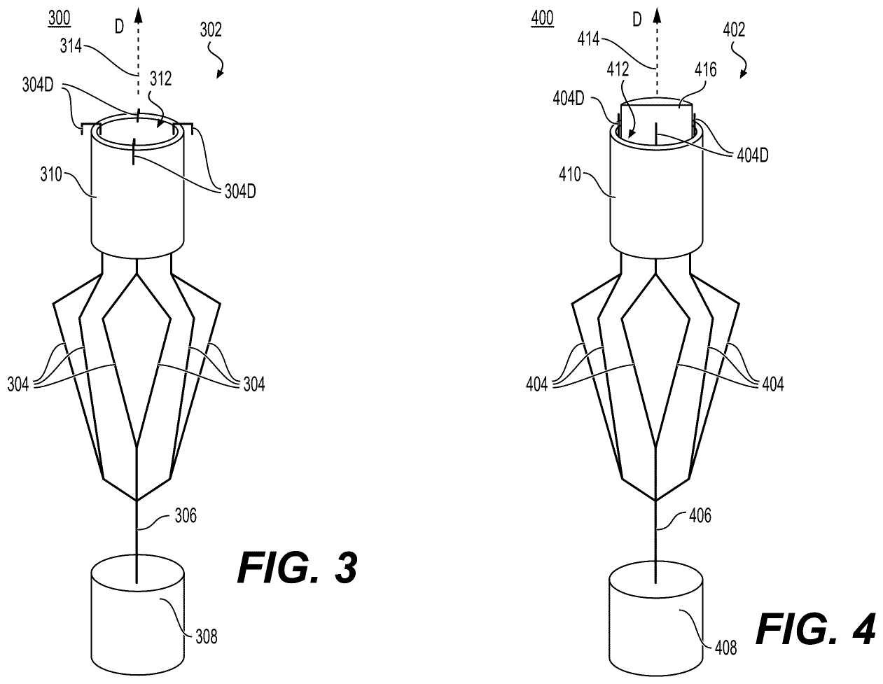

FIG. 3 shows a medical device 300, according to aspects of the disclosure. The medical device 300 may include a retrieval device 302. Aspects of the retrieval device 302 may be similar to aspects of the retrieval device 102 (FIGS. 1-2B), the details of which are omitted for brevity. As shown, the retrieval device 302 includes a plurality of wires 304 (hereinafter, “the wires 304”) having proximal ends coupled to a distal end of a shaft 306 (e.g., at a proximal end of the retrieval device 302), and distal ends coupled to a retention member 310 (e.g., at a distal end of the retrieval device 302). The retrieval device 302 may be movable between at least a closed state and an open state (e.g., radially compress into the closed state and radially expand into the open state). For example, the retrieval device 302 may radially compress into the closed state when positioned within a sheath 308 of the medical device 300 and radially expand when positioned outside of the sheath 308. In the open state, the retrieval device 302 may define a cavity for capturing one or more objects therein.

In some aspects, the retention member 310 of the retrieval device 302 may define a closed distal end of the cavity (e.g., in the open state). The retention member 310 may include, for example, a cannula secured to the wires 304 therein. As shown, the retention member 310 may include an inner surface defining a lumen 312, for example, extending between a proximal end and a distal end of the retention member 310 along a longitudinal axis 314 of the retrieval device 302. The proximal end of the retention member 310 may include a proximal opening extending longitudinally into the lumen 312 toward the distal end, for example, which is shaped and dimensioned to receive distal ends of the wires 304 therein. The distal end of the retention member 310 may include a distal opening extending longitudinally into the lumen 312 toward the proximal end. For example, the wires 304 may include distal portions 304D extending through the distal opening of the retention member 310 and secured to the distal end of the retention member 310.

In some aspects, the distal portions 304D of the wires 304 may include one or more bent portions, or heat set portions, that may help to secure the wires 304 to the retention member 310. In one example, the distal portions 304D may be advanced distally through the lumen 312 of the retention member 310 and bent around the distal end of the retention member 310 to secure the wires 304 therein. In another example, one or more of the wires 304 may be secured to the inner surface defining the lumen 312 of the retention member 310, for example, using one or more fixation or coupling processes (e.g., crimping, welding, soldering, adhesive bonding, etc.). In these aspects, the distal portions 304D of the wires 304 may help to maintain a connection between the retention member 310 and the shaft 306, thus helping to prevent the retention member 310 from being released (e.g., in a body lumen during medical procedures).

In some aspects, it may be useful to cause the retention member 310 to separate or release at least one of the wires 304 of the retrieval device 302, for example, to induce failure in the medical device 300 during medical procedures and release the object(s) captured therein. The medical device 300 may cause the retention member 310 to release one or more of the wires 304, for example, in response to movement of the shaft 306 relative to the sheath 308 and/or movement of the sheath 308 relative to the shaft 306. In one example, proximal translation of the shaft 306 relative to the sheath 308 causes the distal portions 304D of the wires 304 to deform relative to the retention member 310, and in turn allows at least one of the wires 304 to translate proximally through and relative to the retention member 310. For example, the distal portions 304D of the wires 304 may stretch, straighten, or otherwise deform radially inward relative to the retention member 310, such that at least one of the wires 304 can translate proximally through and relative to the retention member 310. In one example, the object(s) captured in the cavity of the retrieval device may impart a force on the retention member 310 in a distal direction relative to the sheath 308, which may at least in part be useful to deform the distal portions 304D of the wires 304 to deform relative to the retention member 310.

In some aspects, the shaft 306 may move (e.g., translate and/or rotate) relative to the sheath 308, which imparts a movement force on the retrieval device 102 that is greater than or equal to a retention force of the retention member 310. The retention force may, for example, be a minimum threshold force between the wires 304 and the retention member 310 which must be overcome to decouple or release at least one of the wires 304 from engagement with the retention member 310. Retention force may be based on one or more aspects of the retrieval device 300, such as the tensile strength of material(s) that form the wires 304, the shape(s) of the distal portions 304D of the wires 304, etc. The movement force may, for example, be sufficient to deform the distal portions 304D of the wires 304 and overcome engagement between the retention member 310 and the wires 304. The movement force may at least in part be, for example, due to contact between the retention member 310 and the object(s) captured in the retrieval device 102. For example, proximal translation of the shaft 306 relative to the sheath 308 may cause the retrieval device 302 to retract proximally into a distal end of the sheath 308, which may cause the object(s) captured therein to move proximally toward and into contact with the distal end of the sheath 308, which may cause the object(s) to contact and impart the movement force on the proximal end of the retention member 310. After contacting the object(s) and the shaft 306 continues moving relative to the sheath 308, the distal portions 304D of the wires 304 may deform in the radially inward direction relative to the retention member 310 and longitudinal axis 314 of the retrieval device 302. After deforming at least one of the wires 304, at least one of the distal portions 304D of the wires 304 may move through and relative to the retention member 310 (e.g., proximal translation through the lumen 312 of the retention member 310).

In alternative implementations, one or more of the wires 304 may be disposed in one or more apertures of the retention member 310 extending into the lumen 312. The wires 304 may be bent, heat set, or otherwise secured in the aperture(s) of the retention member 310. In one example, the wires 304 include distal ends interwoven or threaded through the aperture(s) extending into the lumen 312 between the proximal end and the distal end of the retention member 310.

FIG. 4 shows a medical device 400, according to aspects of the disclosure. The medical device 400 may include a retrieval device 402. Aspects of the retrieval device 402 may be similar to aspects of the retrieval device 102 (FIGS. 1-2B) and/or the retrieval device 302 (FIG. 3), the details of which are omitted for brevity. As shown, the retrieval device 402 may include a plurality of wires 404 (hereinafter, “the wires 404”) having proximal ends coupled to a distal end of a shaft 406 (e.g., at a proximal end of the retrieval device 402), and distal ends coupled to a retention member 410 (e.g., at a distal end of the retrieval device 402).

The retrieval device 402 may be movable between at least a closed state and an open state (e.g., radially compress into the closed state and radially expand into the open state). For example, the retrieval device 402 may radially compress into the closed state when positioned within a sheath 408 of the medical device 400 and radially expand when positioned outside of the sheath 408. In the open state, the retrieval device 402 may define a cavity for capturing one or more objects therein. In some aspects, the retention member 410 of the retrieval device 402 may define a closed distal end of the cavity (e.g., in the open state). The retention member 410 may include, for example, a cannula secured to the wires 404 therein. As shown, the retention member 410 may include an inner surface defining a lumen 412 extending therein, for example between a proximal end and a distal end of the retention member 410 along a longitudinal axis 414 of the retrieval device 402. The proximal end of the retention member 410 may include a proximal opening extending longitudinally into the lumen 412 toward the distal end, for example, which is shaped and dimensioned to receive distal ends of the wires 404 therein. The distal end of the retention member 410 may include a distal opening extending longitudinally into the lumen 412 toward the proximal end.

As shown, the retrieval device 402 may include a plug 416 at least partially disposed in the distal opening of the retention member 410. The plug 416 may be useful help couple the wires 404 within the retention member 410. For example, the wires 404 may include distal portions 404D secured between the inner surface of the retention member 410 and the plug 416 via compression fit. The plug 416 may help to maintain engagement between the retention member 410 and the wires 404 by applying a retention force (e.g., compression fit) on the distal portions 404D of the wires 404. For example, the plug 416 may apply the retention force on the distal portions 404D of the wires 404 disposed between the plug 416 and the retention member 410, for example, radially outward relative to the longitudinal axis 414 of the retrieval device 402.

In some aspects, it may be useful to cause the retention member 410 to separate or release at least one of the wires 404 of the retrieval device 402, for example, to induce failure in the medical device 400 during medical procedures and release the object(s) captured therein. For example, movement of the shaft 406 relative to the sheath 408 may cause movement of the wires 404 relative to the retention member 410, for example, to stretch, straighten, or otherwise deform the distal portions 404D of the wires 404 relative to the retention member 410. The shaft 406 may move relative to the sheath 408, which imparts a movement force that is greater than or equal to the retention force of the plug 416. As the shaft 406 continues to move relative to the sheath 408, the movement force overcomes the retention force of the plug 416 and allows at least one of the wires 404 to move relative to the retention member 410 and the plug 416. In one example, proximal translation of the shaft 406 relative to the sheath 408 causes the distal portions 404D of the wires 404 to deform relative to the retention member 410, which in turn allows at least one of the wires 404 to translate proximally through and relative to the retention member 410 and/or the plug 416.

In alternative implementations, the plug 416 may include one or more slots that are configured to receive at least one of the wires 404 therein. The slot(s) of the plug 416 may be useful to help guide and/or to orient the wires 404 relative to the longitudinal axis 414 of the retrieval device 402. In one example, at least one of the wires 404 is secured within at least one slot of the plug 416 using one or more fixation or coupling processes (e.g., crimping, welding, soldering, adhesive bonding, etc.). In these examples, at least one of the wires 404 is permanently secured to the plug 416 and/or the retention member 410 (e.g., to help prevent the retention member 410 from being released during medical procedures). In another example, at least one of the distal portions 404D of the wires 404 may include one or more bent portions, for example, to help secure at least one of the wires 404 to the plug 416 and/or the retention member 410.

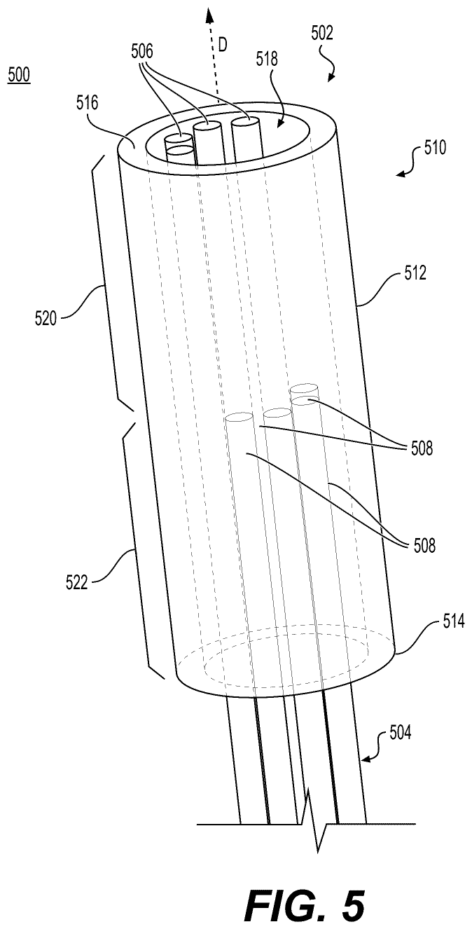

FIG. 5 illustrates a distal portion of a medical device 500, according to aspects of the disclosure. The medical device 500 may include a retrieval device 502. Aspects of the retrieval device 502 may be similar to aspects of the retrieval device 102 (FIGS. 1-2B), the retrieval device 302 (FIG. 3), and/or the retrieval device 402 (FIG. 4), the details of which are omitted for brevity. For instance, the retrieval device 502 may be movable between at least a closed state and an open state (e.g., radially compress into the closed state and radially expand into the open state to capture one or more objects therein). As shown, the retrieval device 502 includes a plurality of wires 504 (hereinafter, “the wires 504”). The wires 504 may include proximal ends coupled to a distal end of a shaft (not shown), and distal ends coupled to a distal tip 510 of the medical device 500. For example, distal tip 510 may be a hypotube. For example, the wires 504 may include distal ends that are secured within the distal tip 510 using one or more fixation or coupling processes (e.g., crimping, welding, soldering, gluing, etc.).

In some aspects, the wires 504 may be grouped into two or more sets of wires 504. As shown, the wires 504 include a first set 506 of wires 504 and a second set 508 of wires 504. The first set 506 and the second set 508 of wires 504 may be respectively coupled to the distal tip 510. The distal tip 510 may receive distal ends of the first set 506 and also receive distal ends of the second set 508 of wires 504 respectively therein, and the distal ends of the wires 504 secured within the distal tip 510 using one or more fixation or coupling processes (e.g., crimping, welding, soldering, gluing, etc.). As shown, the wires 504 include eight (8) wires having distal ends coupled to the distal tip 510, where the first set 506 of wires 504 includes four (4) wires and the second set 508 of wires 504 includes four (4) wires. However, it should be understood the wires 504 may include any number of wires arranged in any number of sets of wires, one or more of which are coupled to the distal tip 510.

In some aspects, the distal tip 510 includes a body 512 defining a lumen 518 extending therein, for example between a proximal end 514 and a distal end 516 of the body 512 along a longitudinal axis of the distal tip 510. The proximal end 514 may include a proximal opening extending longitudinally through the body 512 into the lumen 518 toward the distal end 516 of the distal tip 510. The distal end 516 may include a distal opening extending longitudinally through the body 512 into the lumen 518 toward the proximal end 514 of the distal tip 510. For example, the proximal opening in the proximal end 514 may be shaped and dimensioned to receive distal ends of the first set 506 of wires 504 therein and to receive distal ends of the second set 508 of wires 504 therein.

In some aspects, the first set 506 of wires 504 and/or the second set 508 of wires 504 may include distal ends coupled within the lumen 518 of the distal tip 510 at various positions relative to the longitudinal axis of the distal tip 510. For example, the first set 506 of wires 504 may include distal ends secured within the lumen 518 at a first position, and the second set 508 of wires 504 may include distal ends secured within the lumen 518 at a second position different than the first position relative to the longitudinal axis of the distal tip 510. In one example, the first set 506 of wires 504 include distal ends secured in a distal portion 520 of the distal tip 510, and the second set 508 of wires 504 include distal ends secured in a proximal portion 522 of the distal tip 510. In another example, one wire of the first set 506 of wires 504 has a different length relative to another wire of the first set 506 of wires 504, such that two or more of the first set 506 of wires 504 are respectively secured to the distal tip 510 at different positions relative to the longitudinal axis of the distal tip 510 based on respective wire lengths.

In some aspects, the distal tip 510 includes a filler material or plug (not shown) disposed in the lumen 518 of the distal tip 510, for example, to fill one or more voids in the distal tip 510. For example, filler material may be disposed in the distal portion 520 adjacent to distal ends of the first set 506 of wires 504. The distal end 516 of the distal tip 510 may be closed, for example, to allow filling the body 512 of the distal tip 510 with filler material (e.g., through one or more proximal openings in the proximal end 514).

In some aspects, one or more portions of the distal tip 510 may be exposed to one or more fixation or coupling processes (e.g., crimping, welding, soldering, gluing, etc.) to secure the wires 504 therein. The distal tip 510 may include different portions that are respectively exposed to different fixation processes, for example, to fix the distal tip 510 to one set of wires more securely relative to another set of wires based on respective fixation processes. For instance, in some examples, the distal portion 520 of the distal tip 510 may be exposed to a first fixation process, and the proximal portion 522 of the distal tip 510 may be exposed to a second fixation process different than the first fixation process. In one example, the first set 506 of wires 504 is secured to the distal portion 520 using the first fixation process (e.g., welding) and secured to the proximal portion 522 using the second fixation process (e.g., crimping), and the second set 508 of wires 504 is secured to the proximal portion 522 using the second fixation process (e.g., crimping). In these examples, fixation between the distal tip 510 is stronger or more secure to the first set 506 of wires 504 relative to the second set 508 of wires 504, for example, due to two fixation processes securing the first set 506 and one fixation process securing the second set 508.

In some aspects, it may be useful to induce failure in the medical device 500 during medical procedures, for example, to induce failure in the retrieval device 502 and release one or more objects captured therein. The medical device 500 may cause the distal tip 510 to separate from, or otherwise release, at least one of the wires 504 of the retrieval device 502 (e.g., due to object impaction during medical procedures). In one example, the retrieval device 502 concentrates mechanical stress at the second set 508 of wires 504, thus providing one or more break or separation points along the wires 504 to help induce failure in the retrieval device 502 (e.g., due to object impaction). Breaking the wires 504 or separation the wires 504 from distal tip 510 may open a distal end or side portion of the retrieval device 502 (e.g., in the open state), and in turn release the object(s) captured therein.

In some aspects, it may be useful to form at least one defect (e.g., weak point or portion) in one or more portions of the medical device. The defect may help to provide, for example, a reliable break point in the event of object impaction during a medical procedure. For instance, it may be useful to form one or more defects in a distal tip or distal joint of a retrieval device. The defect(s) may be disposed on the distal tip, such that a portion of the retrieval device reliably breaks at the distal tip. During the medical procedure, in response to object impaction, the distal tip having the defect(s) may break at the position(s) of defect(s) along the distal tip, and in turn cause the retrieval device to release the object.

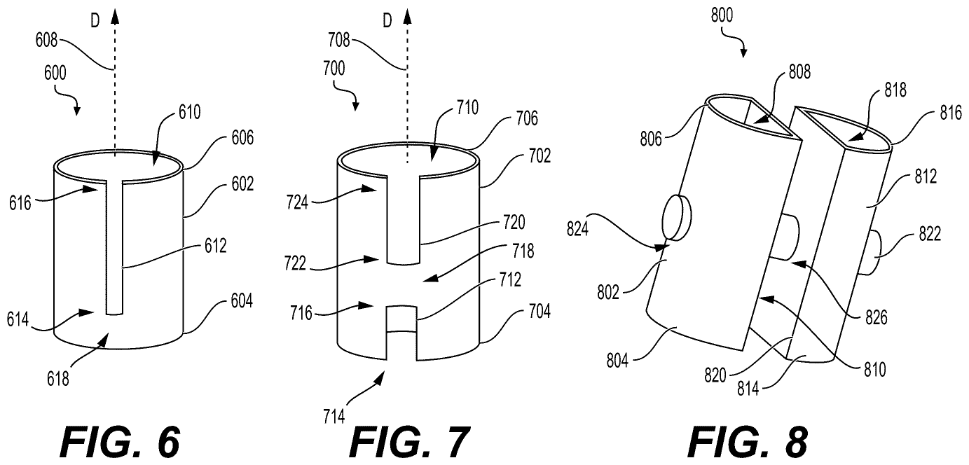

FIG. 6 shows a distal tip 600 of a medical device, according to aspects of the disclosure. The distal tip 600 may include, for example, a hypotube of a retrieval device coupled to a plurality of wires of the retrieval device (not shown). The distal tip 600 may couple distal ends of the plurality of wires together at a distal end of the retrieval device. The distal tip 600 may define a closed distal end of a cavity of the retrieval device, for example, in an open state of the retrieval device to capture one or more objects therein. In some aspects, it may be useful to induce failure in the distal tip 600 during medical procedures, for example, to induce failure in the retrieval device and release the object(s) captured therein.

As shown, the distal tip 600 may include a body 602 extending between a proximal face 604 at a proximal end of the distal tip 600, and a distal face 606 at a distal end of the distal tip 600. The body 602 of the distal tip 600 may include an inner surface that defines a lumen 610 extending therein along a longitudinal axis 608 of the distal tip 600 between the proximal face 604 and the distal face 606. The proximal face 604 may include a proximal opening extending longitudinally through the body 602 into the lumen 610 toward the distal face 606. In one example, the distal face 606 may include a distal opening extending through the body 602 into the lumen 610 toward the proximal face 604. In another example, although not shown, the distal face 606 may form or otherwise define a closed distal end of the lumen 610 extending therein.

In some aspects, the distal tip 600 may be shaped and/or dimensioned to concentrate mechanical stress at one or more positions along the body 602 relative to the longitudinal axis 608. The body 602 of the distal tip 600 may include one or more slots extending therein and defining one or more weak portions of the body 602. For example, the body 602 of the distal tip 600 may include a slot 612. The slot 612 may extend between a first end 614 and a second end 616. The slot 612 may extend radially into the lumen 610 of the body 602 and/or longitudinally through the body 602 approximately parallel to the longitudinal axis 608. As shown, the first end 614 of the slot 612 is positioned between the proximal face 604 and the distal face 606 of the body 602 relative to the longitudinal axis 608, and the second end 616 of the slot 612 is positioned at the distal face 606. The slot 612 may define one or more portions of the body 602 to help induce failure of the distal tip 600 during medical procedures (e.g., due to object impaction). As shown, the distal tip 600 includes a portion 618 of the body 602 positioned between the proximal face 604 and the first end 614 of the slot 612. The distal tip 600 may concentrate mechanical stress at the portion 618 of the body 602 (e.g., defined by the slot 612), thus providing one or more break points to help induce failure of the distal tip 600 (e.g., due to object impaction during medical procedures).

In some aspects, breaking the distal tip 600 releases or otherwise decouples one or more wires of a retrieval device, and in turn releases one or more objects captured in the retrieval device. For example, the distal tip 600 may break or otherwise deform at the portion 618 of the body 602 (e.g., in response to object impaction during medical procedures), which allows one or more wires of the retrieval device to be released or otherwise decoupled from the distal tip 600. This may cause a distal end of the retrieval device to open and release the object(s) captured therein. In one example, the object(s) captured the retrieval device apply force on the proximal face 604 of the distal tip 600 in a distal direction relative to the longitudinal axis 608 of the distal tip 600, which concentrates mechanical stress from the distal force on the portion 618 of the body 602, for example, to break the portion 618 and release one or more wires secured within the lumen 610 of the distal tip 600. In some aspects, the distal tip 600 may be permanently coupled to one or more wires of a retrieval device using one or more fixation or coupling processes (e.g., crimping, welding, soldering, gluing, etc.). In one example, at least one wire of the retrieval device may be secured to an inner surface of the portion 618 of the body 602. In another example, at least one wire of the retrieval device may be secured around the portion 618 of the body 602. This may be useful during medical procedures, for example, to prevent one or more portions of the distal tip 600 (e.g., the portion 618 of the body 602) from being released after object impaction.

FIG. 7 shows another distal tip 700, according to aspects of the disclosure. Aspects of the distal tip 700 may be similar to aspects of the distal tip 600 (FIG. 6), the details of which are omitted for brevity. The distal tip 700 may include, for example, a hypotube of a retrieval device coupled to a plurality of wires of the retrieval device (not shown). The distal tip 700 may couple distal ends of the plurality of wires together at a distal end of the retrieval device. The distal tip 700 may define a distal end of a cavity of the retrieval device, for example, in an open state of the retrieval device to capture one or more objects therein. In some aspects, it may be useful to induce failure in the distal tip 600 during medical procedures, for example, to induce failure in the retrieval device and release the object(s) captured therein.

As shown, the distal tip 700 may include a body 702 extending between a proximal face 704 at a proximal end of the distal tip 700, and a distal face 706 at a distal end of the distal tip 700. The body 702 of the distal tip 700 may include an inner surface that defines a lumen 710 extending therein along a longitudinal axis 708 of the distal tip 700 between the proximal face 704 and the distal face 706. The proximal face 704 may include a proximal opening extending longitudinally through the body 702 into the lumen 710 toward the distal face 706. The body 702 of the distal tip 700 may include a first slot 712 extending between a first end 714 and a second end 716. The body 702 of the distal tip 700 may also include a second slot 720 extending between a first end 722 and a second end 724. In one example, the first end 714 of the first slot 712 may be positioned at the proximal face 704 of the body 702, and the second end 716 of the first slot 712 may be positioned along the body 702 between the proximal face 704 and the distal face 706 relative to the longitudinal axis 708 of the distal tip 700. The first end 722 of the second slot 720 may be positioned along the body 702 between the proximal face 704 and the distal face 706 relative to the longitudinal axis 708 of the distal tip 700, and the second end 724 of the slot 720 may be positioned at the distal face 706 of the body 702. The first slot 712 and the second slot 720 may be circumferentially aligned, or at least partially circumferentially aligned, on the body 702 relative to the longitudinal axis 708 of the distal tip 700.

In some aspects, the distal tip 700 may help to concentrate mechanical stress at one or more positions along the body 702 relative to the longitudinal axis 708. As shown, the body 702 of the distal tip 700 includes a portion 718 positioned between the second end 716 of the first slot 712 and the first end 722 of the second slot 720 relative to the longitudinal axis 708 of the distal tip 700. The distal tip 700 may concentrate mechanical stress at the portion 718 of the body 702 (e.g., defined between the first slot 712 and the second slot 720), thus providing one or more break points to help induce failure of the distal tip 700 (e.g., due to object impaction during medical procedures). Breaking the distal tip 700 releases or otherwise decouples one or more wires of a retrieval device, for example, to release one or more objects captured in the retrieval device. For example, at least one wire of the retrieval device may be secured to an inner surface of the portion 718 of the body 702. In another example, at least one wire of the retrieval device may be secured around the portion 718 of the body 702. This may be useful during medical procedures, for example, to prevent one or more portions of the distal tip 700 (e.g., the portion 718 of the body 702) from being released after object impaction.

FIG. 8 shows another distal tip 800 of a medical device, according to aspects of the disclosure. Aspects of the distal tip 800 may be similar to aspects of the distal tip 600 (FIG. 6) and/or the distal tip 700 (FIG. 7), the details of which are omitted for brevity. The distal tip 800 may include, for example, a split hypotube of a retrieval device coupled to a plurality of wires of the retrieval device that are grouped into two or more sets of wires. For example, the distal tip 800 may respectively receive distal ends of a first set of wires and distal ends of a second set of wires therein, which are respectively secured within the distal tip 800 using one or more fixation processes. The distal tip 800 may respectively couple distal ends of the first set of wires together and couple distal ends of the second set of wires together at a distal end of the retrieval device. The distal tip 800 may define a distal end of a cavity of the retrieval device, for example, in an open state of the retrieval device to capture one or more objects therein. In some aspects, it may be useful to induce failure in the distal tip 800 during medical procedures, for example, to induce failure in the retrieval device and release the object(s) captured therein.

As shown, the distal tip 800 may include a first body 802 defining a first lumen 808 extending therein, for example, between a first proximal face 804 and a first distal face 806. The first proximal face 804 of the first body 802 may include a proximal opening extending longitudinally through the first body 802 into the first lumen 808 toward the first distal face 806 of the distal tip 800. In one example, the proximal opening in the first proximal face 804 of the first body 802 is shaped and dimensioned to receive distal ends of the first set of wires therein. The first distal face 806 may include a distal opening extending longitudinally through the first body 802 into the first lumen 808 toward the first proximal face 804 of the distal tip 800. As further shown, the distal tip 800 may include a second body 812 defining a second lumen 818 extending therein, for example between a second proximal face 814 and a second distal face 816. The second proximal face 814 may include a proximal opening extending longitudinally through the second body 812 into the second lumen 818 toward the second distal face 816 of the distal tip 800. The second distal face 816 may include a distal opening extending longitudinally through the second body 812 into the second lumen 818 toward the second proximal face 814 of the distal tip 800. In one example, the proximal opening in the second proximal face 814 of the second body 812 is shaped and dimensioned to receive distal ends of the second set of wires therein.

In some aspects, the first body 802 of the distal tip 800 may have a shape and dimension configured to or otherwise able to mate with, or otherwise complement, the second body 812 of the distal tip 800. For example, at least one flat surface of the first body 802 may be shaped and dimensioned to mate with at least one flat surface of the second body 812. The rivet 822 may include one or more external features (e.g., grooves, flaps, etc.) that assist with aligning and/or mating complementary surfaces of the first body 802 and the second body 812. In some examples, the first body 802 and the second body 812, respectively, have a semi-circular cross-section shape that are complementary halves of a cylindrical split hypotube (e.g., distal tip 800). As shown, the distal tip 800 may include a first surface 810 of the first body 802 that can mate with a second surface 820 of the second body 812. For example, the first surface 810 of the first body 802 may mate with the second surface 820 of the second body 812 to form a cylinder or at least partially cylindrical shape. In one example, the first surface 810 of the first body 802 extends approximately parallel relative to the second surface 820 of the second body 812. However, it should be understood that the first body 802 and the second body 812 may be oriented at an angle relative to each other and/or to the longitudinal axis of the distal tip 800.

In some aspects, the distal tip 800 may include one or more components, for example, to secure the first body 802 and the second body 812. As shown, the distal tip 800 includes a rivet 822 extending through the first body 802 and the second body 812. The distal tip 800 may include a plurality of apertures extending through the first body 802 and/or the second body 812 and configured to at least partially receive the rivet 822 therein. For example, the rivet 822 may extend through a first pair of apertures 824 formed in the first body 802 and extend through a second pair of apertures 826 formed in the second body 812. The rivet 822 may extend through the first body 802 and/or the second body 812 along a longitudinal axis of the rivet 822 that is oriented at an angle relative to the longitudinal axis of the distal tip 800. For example, the rivet 822 may extend through the first body 802 and the second body 812, such that the longitudinal axis of the rivet 822 is approximately perpendicular to the longitudinal axis of the distal tip 800. However, it should be understood that the rivet 822 may be oriented at other angles relative to the longitudinal axis of the distal tip 800 (e.g., an acute angle). The rivet 822 may be secured to the first body 802 and the second body 812, such that the first body 802 is separated from the second body 812 relative to the longitudinal axis of the distal tip 800 (e.g., an air gap is disposed horizontally therebetween).

In some aspects, it may be useful to induce failure in the distal tip 800 during medical procedures, for example, to induce failure in the rivet 822 of the distal tip 800 and release the object(s) captured within the retrieval device. Breaking the rivet 822 of the distal tip 800 may cause the first body 804 and the second body 812 to separate and thereby to open the distal end of the cavity of the retrieval device defined by the distal tip 800. In one example, the rivet 822 may have a variable cross-section (e.g., diameter) along a length of the rivet 822. The variable cross-section of the rivet 822 may define at least one defect (e.g., weak point or portion) along the length of the rivet 822. The defect provides, for example, a reliable break point in the event of object impaction during medical procedures. In one example, the rivet 822 includes a thinner portion disposed between the first body 802 and the second body 812 when the distal tip 800 is assembled. The distal tip 800 may concentrate mechanical stress at the thinner portion of the rivet 822, thus providing one or more break points to help induce failure of the distal tip 800 (e.g., due to object impaction during medical procedures). In one example, inducing failure in the rivet 822 may include proximally translating one or more wires secured within the distal tip 800 relative to the rivet 822, which may cause the rivet 822 to break or at least partially deform such that the captured object(s) may be released.

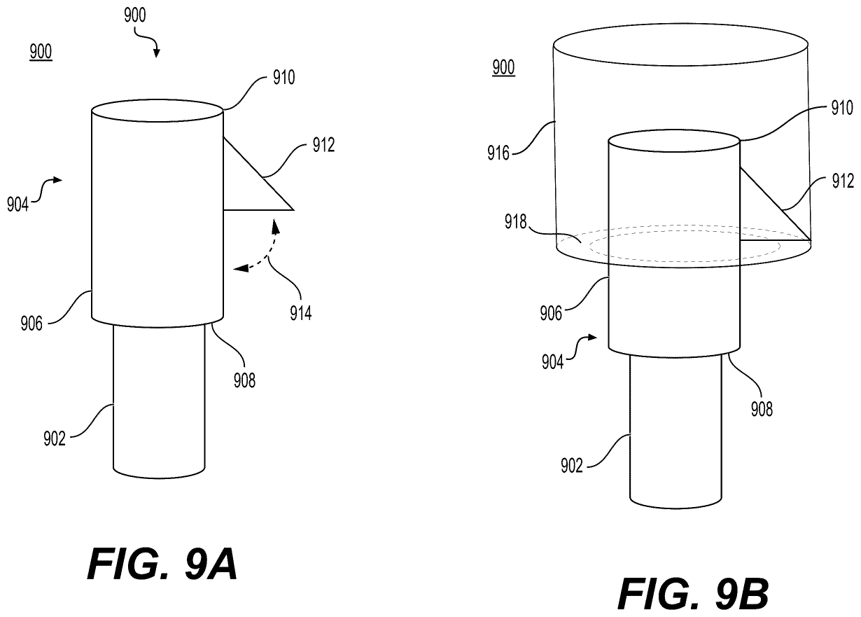

FIG. 9A shows a distal portion of a medical device 900, according to aspects of the disclosure. Aspects of the medical device 900 may be similar to aspects of other medical devices discussed herein, the details of which are omitted for brevity. The medical device 900 may include a retrieval device including at least one wire 902 coupled to a tip 904. The tip 904 may include, for example, a wire tip coupled to distal ends of the at least one wire 902. In one example, at least one wire 902 of the medical device 900 includes a distal end of a single wire of the retrieval device. In another example, at least one wire 902 of the medical device 900 includes distal ends of a plurality of wires of the retrieval device. In another example, at least one wire 902 of the medical device 900 includes a sheath disposed circumferentially about a plurality of wires of the retrieval device.

As shown, the tip 904 may include a body 906 extending between a proximal surface 908 at a proximal end of the tip 904, and a distal surface 910 at a distal end of the tip 904. The body 906 may include an inner surface defining a lumen extending therein, for example between the proximal surface 908 and the distal surface 910 along a longitudinal axis of the tip 904. The proximal surface 908 of the tip 904 may include a proximal opening extending longitudinally through the body 906 into the lumen toward the distal surface 910. The proximal surface 908 of the tip 904 may be configured to receive the at least one wire 902 therein and secured in the tip 904 using one or more fixation processes (e.g., crimping, soldering, welding, adhesive bonding, etc.). In other examples, the tip 904 may be integrally formed with the at least one wire 902.

In some aspects, it may be useful to couple distal ends of at least one wire 902 of the medical device 900 to a distal tip of the retrieval device, for example a hypotube or crimping sleeve (not shown). As shown, the tip 904 may include a retention member, element, or flap 912. The retention flap 912 may be configured to engage a distal tip (e.g., hypotube or crimping sleeve) and to secure the at least one wire 902 to the distal tip. For example, the tip 904 may be inserted distally through a proximal opening of a hypotube and secured within a lumen of the hypotube via the retention flap 912. The retention flap 912 includes at least one surface having shaped and dimensioned to engage one or more inner surfaces of the hypotube, such as a groove or recess formed in an inner surface defining the lumen of the hypotube which engages the retention flap 912 of the tip 904 therein. This engagement may help to retain the tip 904 in the lumen of the hypotube, for example, to assemble a retrieval device of the medical device 900 for capturing one or more objects therein during medical procedures. Although the retention flap 912 is shown as one component in FIG. 9A, it should be understood that the tip 904 of the medical device 900 may include a plurality of retention flaps 912 extending from, and/or movable relative, to the body 904.

In one example, the retention flap 912 may include a fixed protrusion extending radially outward from the body 904 relative to the longitudinal axis of the distal tip 904. In another example, the tip 904 may be movable between at least a closed state and an open state. For instance, the retention flap 912 may pivot relative to the body 904 about a pivot point 914 in a radially outward direction and/or a radially inward direction relative to the longitudinal axis of the tip 904. In another example, the retention flap 912 may move from the closed state into the open state of the tip 904, for example, to engage the distal tip of the retrieval device and secure the at least one wire 902 to the distal tip.

In some aspects, it may be useful to induce failure in the medical device 900 during medical procedures, for example, to induce failure in the tip 904 of the retrieval device and release the object(s) captured therein. The medical device 900 may be configured to break or deform the retention flap 912 of the tip 904, and in turn release the at least one wire 904 secured therein. For example, the medical device 900 may concentrate mechanical stress at the retention flap 912 of the tip 904. Applying force greater than or equal to a threshold force may cause the retention flap 912 to break or disengage from the body 904 and thus induce failure of the medical device 900.

FIG. 9B illustrates the medical device 900 of FIG. 9A, according to aspects of the disclosure. As shown, the medical device 900 may include a tubular element or member, for example, a hypotube 916 coupled to the tip 904. The hypotube 916 may define a lumen extending therein, for example between a proximal end and a distal end of the hypotube 916. The hypotube 916 may include one or more proximal openings at the proximal end that are configured to receive one or more of the tip 904 therein. As shown, the tip 904 is disposed at least partially within the hypotube 916, such that the retention flap 912 may engage an inner surface 918 of the hypotube 916. This engagement may help to retain the tip 904 within the hypotube 916, for example, to define a distal end of the medical device 900 in the open state.

In an alternative implementation, the tip 904 may include a biasing member (not shown) configured to bias the retention flap 912, for example, radially outward and/or radially inward relative to the body 906. For example, the tip 904 may include one or more springs that are coupled to the retention flap 912 and bias the retention member 812 radially outward relative to the body 906. The body 906 may include a slot formed therein which may receive the retention flap 912 therein, such that the retention flap 912 is movable at least between a closed state (e.g., retracted within the lumen) and an open state (e.g., extended radially outward). The retention flap 912 may extend longitudinally between a first end secured to the body 906 (e.g., distal end) and a second end (e.g., proximal end) secured to the biasing member (e.g., spring). The biasing member may be secured to the second end of the retention flap 912, such that the biasing member may displace the second end radially outward relative to the first end of the retention flap 912.

In another alternative implementation, the medical device 900 may include a distal tip having a plurality of channels extending therein. Each channel may have a shape and dimension configured to receive the tip 904 at least partially therein. For example, the distal tip may include eight channels collectively configured to receive eight of the tip 904 therein. Each tip 904 may be coupled to a respective wire of a plurality of wires (e.g., eight wires coupled to each tip 904). In one example, at least one tip 904 is permanently attached to the distal tip (e.g., to prevent shedding).

In another alternative implementation, the tip 904 may include two or more of the retention flap 912. For example, the tip 904 may include two of the retention flap 912 that are circumferentially spaced relative to the body 906. Each of the retention flap 912 may engage the inner surface 918 of the hypotube 916 which, for example, may help to retain the tip 904 within the hypotube 916.

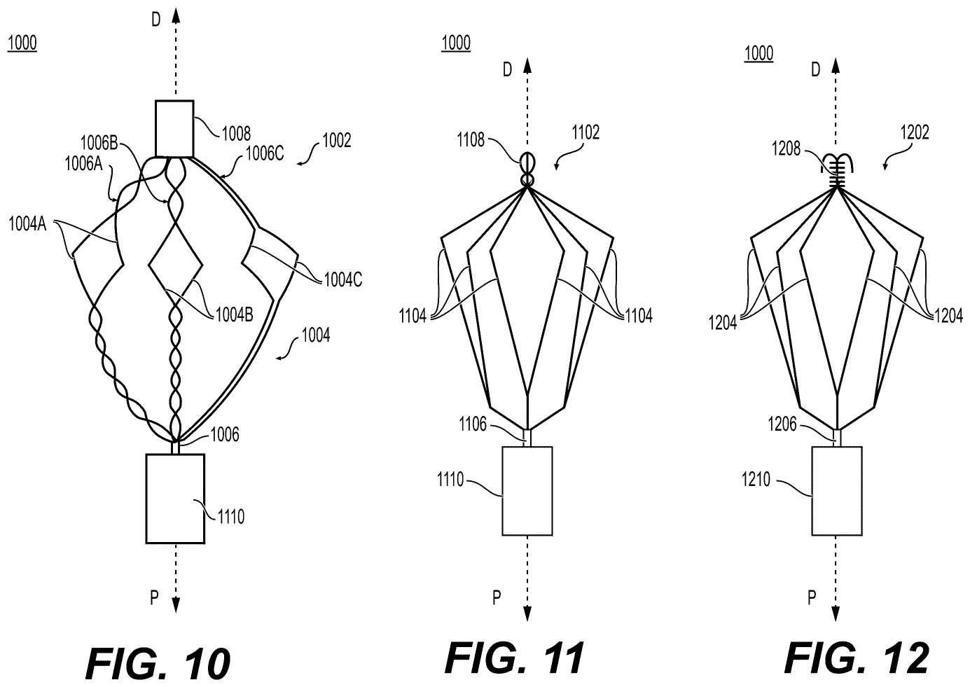

FIG. 10 shows a medical device 1000, according to aspects of the disclosure. The medical device 1000 may include a retrieval device 1002. Aspects of the retrieval device 1002 may be similar to one or more aspects of retrieval devices discussed herein, the details of which are omitted for brevity. As shown, the retrieval device 1002 includes a plurality of wires 1004 (hereinafter, “the wires 1004”) extending between a proximal end and a distal end of the retrieval device 1002. The wires 1004 may include proximal ends coupled to a distal end of a shaft 1006 at the proximal end, and distal ends coupled to a distal tip 1008 at the distal end. The shaft 1006 may at least partially disposed in a sheath 1010 of the medial device 1000. The shaft 1006 and the sheath 1010 may move (e.g., translate and/or rotate) relative to each other, for example, to move the retrieval device 1002 between at least a closed state and an open state (e.g. radially compress into the closed state when positioned in the sheath 1010, and radially expand into the open state when positioned outside of the sheath 1010).

In some aspects, it may be useful to form at least one defect in the retrieval device 1002, which may help to provide, for example, a reliable break point in the event of object impaction during a medical procedure. The wires 1004 may be grouped into two or more sets, such that at least one set of the wires 1004 includes at least one defect to provide at least one reliable break point to induce failure of the retrieval device 1002. In one example, the wires 1004 of the retrieval device 1002 may be grouped into a first set 1004A of wires 1004, a second set 1004B of wires 1004, and a third set 1004C of wires 1004. As shown, the first set 1004A of wires 1004 have one or more twisted portions 1006A intertwined or twisted along a length thereof, and the second set 1004B of wires 1004 have one or more twisted portions 1006B intertwined or twisted along a length thereof. The third set 1004C of wires 1004 have one or more untwisted portions 1006C that are not intertwined or twisted along a length thereof. The retrieval device 1002 may concentrate mechanical stress at the untwisted portions 1006C of the third set 1004C of wires 1004 (e.g., during medical procedures due to object impaction), which have decreased strength relative to the twisted portions 1006A of the first set 1002A of wires 1004 and the twisted portions 1006B of the second set 1002B of wires 1004. The retrieval device 1002 helps to concentrate mechanical stress on the untwisted set of the wires 1004, thus providing one or more reliable break points along the wires 1004 to help induce failure (e.g., due to object impaction during medical procedures).

FIG. 11 shows a medical device 1100, according to aspects of the disclosure. The medical device 1100 may include a retrieval device 1102. Aspects of the retrieval device 1102 may be similar to aspects of other retrieval devices discussed herein, the details of which are omitted for brevity. As shown, the retrieval device 1102 may include a plurality of wires 1104 (hereinafter, “the wires 1104”) extending between a proximal end and a distal end of the retrieval device 1102. The wires 1104 may include proximal ends coupled to a distal end of a shaft 1106 of the medical device 1100. The shaft 1106 may at least partially disposed in a sheath 1110 of the medial device 1100. The shaft 1106 and the sheath 1110 may move (e.g., translate and/or rotate) relative to each other.

In some aspects, the wires 1104 include distal ends that are shaped and/or interlocked to form a distal tip 1108 of the retrieval device 1102. The distal tip 1108 may define a closed distal end of a cavity defined by the retrieval device 1102 in the open state (e.g., when positioned outside of the sheath 1110). The wires 104 may include a first set of wires that are interlocked around a second set of wires to define the distal tip 1108. For example, the wires 1104 may include a first set of three (3) wires interlocked around a second set of five (5) wires to define the distal tip 1108.

In some aspects, movement of the medical device 1100 may be configured to unfurl or otherwise separate the interlocked distal ends of the wires 1104 that define the distal tip 1108. For example, with one or more objects within the wires 1104 proximal translation of the shaft 1106 relative to the sheath 1110 may help to separate the interlocked distal ends of the wires 1104, which in turn opens a distal end of the retrieval device 1102 to release one or more objects captured therein. For example, when the shaft 1106 proximally translates relative to the sheath 1110, this movement helps to cause the object(s) captured within the retrieval device 1102 to apply force on the distal tip 1108. The force due to proximal translation may overcome a retention force of the distal tip 1108, thus causing interlocked distal ends of the wires 1104 to separate, which may help to release the object(s) from the retrieval device 1102.

FIG. 12 shows another medical device 1200, according to aspects of the disclosure. The medical device 1200 may include a retrieval device 1202. Aspects of the retrieval device 1202 may be similar to aspects of other retrieval devices discussed herein, the details of which are omitted for brevity. As shown, the retrieval device 1202 may include a plurality of wires 1204 (hereinafter, “the wires 1204”) having proximal ends coupled to a distal end of a shaft 1206 of the medial device 1200. The shaft 1204 may be disposed in, and move relative to, a sheath 1210 of the medial device 1200.

In some aspects, the wires 1204 include distal portions that are hooked together to form a hooked distal tip 1208 of the retrieval device 1202. The wires 1204 may include a first set of wires that are hooked to or otherwise secured to a second set of wires, such that distal ends of the wires 1204 form the hooked distal tip 1208 at a distal end of the retrieval device 1202. The wires 1204 may be “shape-set” to form the hooked distal tip 1208, for example, by exposing distal ends of the wires 1204 to energy or heat for an extended period of time while oriented in a desired shape of the hooked distal tip 1208 (e.g., hook shape). For example, as shown in FIG. 12, the wires 1204 include a first set of the wires 1204 that are hooked around a second set of the wires 1204 to form the hooked distal tip 1208. In one specific example, the wires 1204 include a first set of two (2) wires 1204 hooked around a second set of five (5) wires 1204 and form the hooked distal tip 1208. Although it should be understood that each set of wires may have more or fewer of the wires 1204 to form the hooked distal tip 1208.

In some aspects, it may be useful to unfurl or otherwise separate one or more portions of the distal tip 1208 during medical procedures. In the open state, the hooked distal tip 1208 defines a closed distal end of a cavity of the retrieval device 1202 (e.g., to capture objects therein). It may be useful to open the cavity of the retrieval device 1202, for example, to help release captured object(s) therein. In one example, the hooked distal tip 1208 may unfurl in response to movement of the shaft 1206 relative to the sheath 1210. In another example, the hooked distal tip 1208 may unfurl in response to objection impaction. In yet another example, proximal translation of the shaft 1206 relative to the sheath 1210 may cause the hooked distal tip 1208 to be driven or otherwise moved into contact with the object(s) in the cavity of the retrieval device 1202 which imparts impaction force distally on the hooked distal tip 1208. In turn, this may cause the hooked distal tip 1208 to unfurl.

Each of the aforementioned devices, assemblies, systems, and related methods may be used for medical procedures to help extract one or more objects from a patient. By providing a medical device capable of capturing and releasing an object as discussed herein, known problems associated with extraction medical procedures and/or other aspects of invasive surgical procedures may be reduced or avoided. For example, various aspects of this disclosure may help to provide a retrieval device that is able to capture the object and to release the object during a medical procedure. Additionally, providing a medical device that includes a tether coupled to a sheath and a retrieval device may be useful, for example, to help prevent or mitigate component shedding in the patient during medical procedures. Based on these aspects, physicians, or other users of may reduce the overall procedure time, increase efficiency of procedures, and/or avoid unnecessary harm to a patient's body during extraction medical procedures.

It will be apparent to those skilled in the art that various modifications and variations may be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A medical device, comprising:
a sheath;
a shaft, wherein at least a portion of the shaft is movably disposed within the sheath; and
a retrieval device coupled to a distal end of the shaft, wherein the retrieval device is movable between at least a closed state and an open state, wherein the retrieval device is configured to radially compress into the closed state when positioned within the sheath, and wherein the retrieval device is configured to radially expand into the open state when positioned outside of the sheath, wherein the retrieval device comprises:
a plurality of wires, wherein proximal ends of the plurality of wires are coupled to the distal end of the shaft;
a retention member coupled to distal ends of the plurality of wires;
wherein movement of the shaft relative to the sheath is configured to release one or more of the plurality of wires from the retention member of the retrieval device.

2. The medical device of claim 1, wherein the retention member comprises a cannula that includes a body having a lumen extending therein along a longitudinal axis, wherein the plurality of wires is coupled at least partially within the lumen.

3. The medical device of claim 2, further comprising a plug configured to be received within the lumen of the cannula, and wherein distal ends of the plurality of the wires are coupled within the lumen of the cannula between the plug and the body of the cannula.

4. The medical device of claim 2, wherein distal ends of the plurality of wires engage the body of the cannula at an angle relative to the longitudinal axis of the cannula.

5. The medical device of claim 4, wherein distal ends of the plurality of wires are bent over a distalmost end of the body of the cannula.

6. The medical device of claim 1, wherein the retention member comprises a coil disposed around the plurality of wires.

7. The medical device of claim 6, wherein proximal translation of the shaft relative to the sheath causes the coil to release one or more of the plurality of wires.

8. The medical device of claim 6, wherein the retention member is welded to at least one of the plurality of wires.

9. The medical device of claim 6, wherein the coil is disposed around distal ends of the plurality of wires in a z-shaped configuration.

10. The medical device of claim 1, wherein the plurality of wires comprises a first set of wires and a second set of wires, wherein distal ends of the first set of wires are coupled together, and wherein distal ends of the second set of wires are coupled together.

11. The medical device of claim 10, wherein the plurality of wires comprises a third set of wires and a fourth set of wires, wherein distal ends of the third set of wires are coupled together, and wherein distal ends of the fourth set of wires are coupled together.

12. The medical device of claim 10, wherein the retention member is welded to at least one of the first set of wires or the second set of wires of the plurality of wires.

13. The medical device of claim 1, further comprising a tether having a distal portion coupled to the retention member and a proximal portion coupled to one of the sheath and the shaft.

14. The medical device of claim 13, wherein the tether comprises at least one of a polymer material and a metal material.

15. The medical device of claim 1, wherein the plurality of wires radially expands into the open state to define a cavity configured to capture an object therein.

16. A medical device, comprising:
a sheath;
a shaft, wherein at least a portion of the shaft is movably disposed within the sheath; and
a retrieval device coupled to a distal end of the shaft, wherein the retrieval device comprises:
a plurality of wires, wherein proximal ends of the plurality of wires are coupled to the distal end of the shaft;
a distal tip coupled to distal ends of the plurality of wires, wherein the distal tip comprises:
a body having a lumen extending therein along a longitudinal axis between a proximal end and a distal end; and
at least one slot extending through the body along the longitudinal axis from one of the proximal end of the body and the distal end of the body.

17. The medical device of claim 16, wherein the slot extends from the distal end of the body proximally toward the proximal end of the body.

18. The medical device of claim 16, wherein the slot includes a first slot and a second slot, wherein the first slot extends distally from the proximal end of the body, and wherein the second slot extends proximally from the distal end of the body toward a distal end of the first slot.

19. A medical device, comprising:
a sheath;
a shaft, wherein at least a portion of the shaft is movably disposed within the sheath; and
a retrieval device coupled to a distal end of the shaft, wherein the retrieval device is movable between at least a closed state and an open state, wherein the retrieval device is configured to radially compress into the closed state when positioned within the sheath, and wherein the retrieval device is configured to radially expand into the open state when positioned outside of the sheath, wherein the retrieval device comprises:
a plurality of wires, wherein proximal ends of the plurality of wires are coupled to the distal end of the shaft;
a hypotube coupled to distal ends of the plurality of wires,
wherein the plurality of wires comprises a first set of wires and a second set of wires, wherein distal ends of the first set of wires are secured within a distal portion of the hypotube, and wherein distal ends of the second set of wires are secured within a proximal portion of the hypotube.

20. The retrieval device of claim 19, wherein the first set of wires are welded to the distal portion of the hypotube, and wherein the second set of wires are secured to the hypotube via a crimp.
